(19) United States
(12) Patent Application Publication
(10) Pub. No.: US 2012/0095431 A1
(43) Pub. Date: Apr. 19, 2012

(54) DIAPER WITH IMPROVED CONJUGATION STRUCTURE

(76) Inventors: Po-An TSAI, Kaohsiung (TW); Charng-Ching OU, Taipei (TW); Hsu-Feng SHIH, Changhua (TW)

(21) Appl. No.: 13/163,778

(22) Filed: Jun. 20, 2011

(30) Foreign Application Priority Data
Oct. 14, 2010 (TW) TW099219834

(51) Int. Cl. A61F 13/62

(52) U.S. Cl. 604/391

(57) ABSTRACT

A diaper having an improved conjugated structure that is convenient, low-cost and easy for mass production is disclosed. The diaper includes a side wing having a magic hook located thereon and a rear thin sheet having an anti-leaking layer and a non-textile fabrics layer conjugated entirely or partially. The conjugation strength between the magic hook and the rear thin sheet is about 100 to 700 g/inch at 180 degrees, and the shear stress at 180 degrees is over 1000 g/inch. With the conjugation between the magic hook and the rear thin sheet, the user can randomly attach the magic hook to the surface of the non-textile fabrics layer to adjust the tightness of the diaper.

### FIELD OF THE INVENTION

The present invention is related to a diaper, and more specifically to a diaper with an improved conjugation structure.

### BACKGROUND OF THE INVENTION

Japanese people were the first trying to find substitution for a diaper since people started to use it, because there is very little natural resource in Japan. In 1942, Pauli Strom in Sweden invented a diaper with two pieces: an outer layer that is a plastic pant and an inner layer that is an absorption pad made by paper. This kind of diaper is easily broken and the scrapes will stick on the baby's bottom, so it has not been widely used for a long time.

In 1997, the Time magazine elected one of the one hundred greatest inventions in the twentieth century to be Pampers diaper, invented by P&G. There was a story that the reason why P&G would invent Pampers is because an R&D manager had a granddaughter and he was annoyed by repeatedly changing his granddaughter's diapers, so he made up his mind to replace conventional diapers with a substitute that does not have to be washed.

Currently in the development of diapers, there are only two ways to secure the diaper on the baby's waist. First, a traditional tape (Tape) conjugated with PP or PE or PET films (Frontal): a user attaches the sticky Tape to the PP film to secure the diaper on the baby's waist. The advantage is the cost of the materials is cheaper, while it is disadvantageous that when the user has too much moisture, bath powder or baby lotion on the hands and accidentally touches the upper surface of the Tape or Frontal, the securing functionality can be significantly reduced or there will be no more securing functionality, such that the diaper may have to be discarded because it cannot be secured at the baby's waist. The anti-leaking membrane of the diaper is PE (polyethylene) and if the user accidentally put the Tape on the PE film, the Tape cannot be separated from the PE film. If the user exerts more power to separate them, the surface of the PE film will be broken.

Second, a so-called VELCRO that uses a magic hook (Hook) conjugated with PP or PE surface, or a mesh surface (Loop). The principle to secure the diaper is similar to that mentioned above: Hook is attached to the Loop that may be a small and restricted area. The advantage is that when the user has too much moisture, bath powder or baby lotion, the securing functionality is not affected, while it is disadvantageous that the cost of materials and manufacturing is more expensive.

Thus, although the second method stated above has already eliminated the disadvantages and is more convenient for the consumers, the price is still too high so that the second method is still less popular than the first method that uses Tape conjugated with PP Frontal.

Therefore, there remains a need for a new and improved conjugation structure of the diaper that can not only reduces the usage of materials (namely reduces the cost), but also increases the manufacturing yield.

### SUMMARY OF THE INVENTION

The present invention is to provide an improved conjugation structure of a diaper that is convenient, low-cost and easy for mass production.

The diaper with improved conjugation structure in the present invention includes a diaper (100) having a front waistline segment (1) located along vertical direction, a rear waistline segment (2) located along vertical direction facing the front waistline segment (1), and a crotch segment (3) located along horizontal direction and between the front waistline segment (1) and the rear waistline segment (2).

One side of the diaper (100) contacting the skin has a liquid-penetrating surface sheet (4), and a rear thin sheet (5), which cannot be penetrated by liquid, is located at a side where the diaper (100) does not contact the skin. Also, a liquid absorption unit (6) extending between the front waistline segment (1) and the rear waistline segment (2) is located between the surface sheet (4) and the rear thin sheet (5); and a pair of rear wings (7) is extending from the liquid absorption unit (6) toward the horizontal direction while a pair of side wing (8) is extending from the liquid absorption unit (6) toward the vertical direction, wherein a magic hook (9) is located at each side wing (8) and extending towards the horizontal direction.

The rear thin sheet (5) is formed by an anti-leaking layer (51) from the top and a non-textile fabrics layer (52) which are coupled entirely or partially. The strength to separate the magic hook (9) and the rear thin sheet (5) at 180 degrees is about 100 to 700 g/inch, and the shear stress at 180 degrees is above 1000 g/inch. With the conjugation between the magic hook (9) and the rear thin sheet (5), the user can randomly attach the magic hook (9) to the surface of the non-textile fabrics layer (52) to adjust the tightness of the diaper (100).

The surface of the rear thin sheet (5) can be one of the following: a velvet surface, a fabric surface or a rough surface. With the changes of the surface of the rear thin sheet (5), the present invention can be different in appearance and touch, as well as increasing the stickiness between the surface and the magic hook (9).

The surface of the non-textile fabrics layer (52) has a plurality of three-dimensional drawings (521) that are continuously and regularly formed on the surface of the non-textile fabrics layer (52). In other embodiments, the three-dimensional drawings (521) can be discontinuously and irregularly formed on the surface of the non-textile fabrics layer (52). In still other embodiments, the three-dimensional drawing (521) is one of the following: decorative patterns, dots, squares and regular stripes.

When the baby wears the diaper (100), the most outer surface of the diaper (100), namely the non-textile fabric layer (52) of the rear thin sheet (5), has the three-dimensional drawings (521) which are protruding or recessed, so the surface of the rear thin sheet (5) has protruding-recessed lines like other knit products, which can not only increase the diaper's sense of touch, but also provide better feeling to the parents especially when they hold the baby. The parents can directly feel gentle sense of touch from the three-dimensional drawings (521) on the non-textile fabric layer (52).

Furthermore, in addition to enhancing the sense of touch by adding the three-dimensional drawings (521), it can further strengthen the holding power between the anti-leaking layer (51) and the non-textile fabric layer (52), which can not only increase the aesthetic value, but also increase the conjugation between the non-textile fabric layer (52) and the magic hook (9) to increase the possibility of reuse.

On the surface of the anti-leaking layer (51)/non-textile fabric layer (52), there are either a combination of a regular mark (54) and a mark in the target area (55), or each mark individually. Even though it is already very convenient for the user by conjugating the magic hook (9) and the rear thin sheet (5), the user has been using conventional diapers for a long time. Therefore, in order to attract more consumers, in addition to adding more colorful marks, trademarks, or other marks with special design, the mark in the target area (55) can also be added to replace the area replaced by Loop. However, if the budget is not allowed, there can be no regular mark (54), nor the mark in the target area (55).

The anti-leaking layer (51) and the non-textile fabrics layer (52) are made by elastic materials and the elastic coefficients of the anti-leaking layer (51) and the non-textile fabrics layer (52) are different. Also, with the combination of the anti-leaking layer (51) and the non-textile fabrics layer (52), the present invention can reduce the baby's uncomfortable feeling and fit the baby's body so it is easier for the baby to move.

In one embodiment, the anti-leaking layer (51) is made of the following materials: breathable and non-breathable. More specifically, the anti-leaking layer (51) is made of one of the following materials: PP (polypropylene), PE (polyethylene), copolymerized composite materials. The material of the anti-leaking layer (51) can be determined by the application of the diaper (100).

Comparing with conventional techniques, the present invention has the following advantages: (I) Using the non-textile fabrics layer (52) to be the basic material of the rear thin sheet (5) can not only serve the economical purpose, but also solve the problem of material overuse to achieve the goal of saving resources. (II) Utilizing the strength of the non-textile fabrics layer (52) itself to combine with the anti-leaking layer (51) and then utilizing the physical strength of the non-textile fabrics layer (52) to replace the usage of Loop can lower the manufacturing costs of the diaper. (III) Due to the combination of the anti-leaking layer (51) and the non-textile fabrics layer (52), the diaper manufacturer can significantly lower the manufacturing costs of the diaper and may no longer need the Loop. (IV) With the combination of the anti-leaking layer (51) and the non-textile fabrics layer (52), the manufacturers can avoid the problems such as misalignment, non-stickiness, misplacement and excessive glue because there is no Loop on the diaper, such that the quality of the diaper can be significantly increased. (V) For the consumer purchasing the diaper (100), with the combination of the anti-leaking layer (51) and the non-textile fabrics layer (52) along with the magic hook (9), it is more convenient for the consumer because the area/surface which can be used for Loop is significantly increased so the consumer can randomly adjust the position and tightness whenever they want to provide a comfortable way for a baby's waist. Moreover, since the waist area of the diaper (100) can be adjusted in a broad range, the consumer can purchase any size of the diaper (100) so the flexibility is increased to avoid potential waste. (VI) With the combination of the anti-leaking layer (51) and the non-textile fabrics layer (52), it reduces the usage of the materials to further protect the environment. A diaper may require several kinds of materials and several different methods to put the materials together. In modern high-speed manufacturing process, it is not an easy task to put together that many different materials to form a diaper. In other words, many process steps may be needed. Therefore, in addition to replacing Loop, the present invention can also simplify the manufacturing process to avoid mistakes occurred during the process, so that the yield can be increased and the usage of materials can be reduced to further achieve the goal of environmental protection. Also, with the combination of the anti-leaking layer (51) and the non-textile fabrics layer (52), the strength of the conjugation is as strong as the replaced Loop. As to the diaper manufacturers, it is convenient to process and saves the materials. More importantly, the consumer does not feel uncomfortable and on the other hand, the present invention is closer to the consumer's style of using the diaper. Furthermore, with the combination of the anti-leaking layer (51) and the non-textile fabrics layer (52), it reduced the usage of the anti-leaking layer (51) with higher unit price so the costs are reduced as well.

The combination of the anti-leaking layer (51) and the non-textile fabrics layer (52) can be entirely or partially, wherein the partial combination is processed externally so that the designated portion of the diaper can be strengthened, and the strength is increased at the entire area of the diaper (100) or the small area on the Loop. The increase in strength is to strengthen and replace conventional Loop and the area and shape of the external process is not restricted by the drawing or area.

### BRIEF DESCRIPTION OF THE DRAWINGS

FIG. 1 illustrates a three-dimensional schematic view in the present invention.

FIG. 2 illustrates a partial sectional view of FIG. 1 in the present invention.

FIG. 3 illustrates a schematic view of one embodiment in the present invention.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

In order to further understand the goal, characteristics and effect of the present invention, a number of embodiments along with the drawings are illustrated as following:

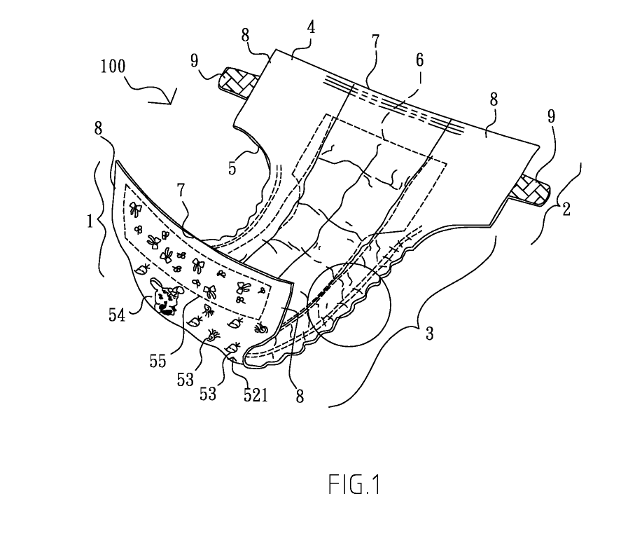

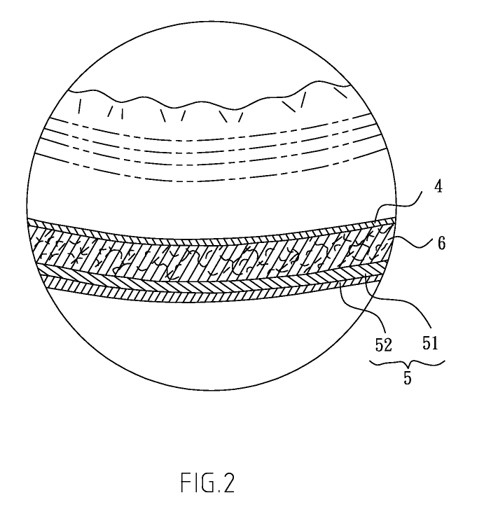

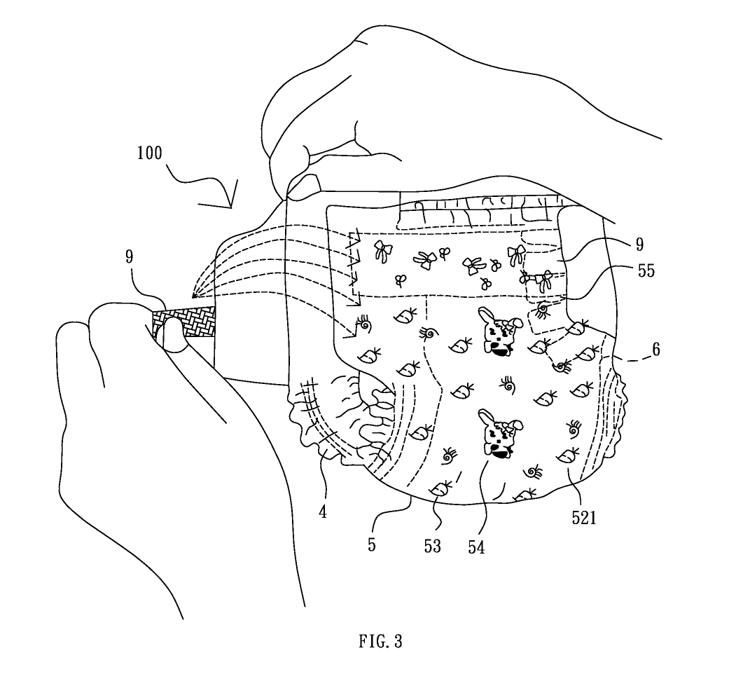

Referring to FIGS. 1 to 3, the present invention provides a diaper with an improved conjugation structure, including a diaper (100) having a front waistline segment (1) located along vertical direction, a rear waistline segment (2) located along vertical direction facing the front waistline segment (1), and a crotch segment (3) located along horizontal direction and between the front waistline segment (1) and the rear waistline segment (2).

One side of the diaper (100) contacting the skin has a liquid-penetrating surface sheet (4), and a rear thin sheet (5), which cannot be penetrated by liquid, is located at a side where the diaper (100) does not contact the skin. Also, a liquid absorption unit (6) extending between the front waistline segment (1) and the rear waistline segment (2) is located between the surface sheet (4) and the rear thin sheet (5); and a pair of rear wings (7) is extending from the liquid absorption unit (6) toward the horizontal direction while a pair of side wing (8) is extending from the liquid absorption unit (6) toward the vertical direction, wherein a magic hook (9) is located at each side wing (8) and extending towards the horizontal direction.

The rear thin sheet (5) is formed by an anti-leaking layer (51) from the top and a non-textile fabrics layer (52) which can be coupled with the anti-leaking layer (51) entirely or partially. The strength to separate the magic hook (9) and the rear thin sheet (5) at 180 degrees is about 100 to 700 g/inch, and the shear stress at 180 degrees is above 1000 g/inch. With the conjugation between the magic hook (9) and the rear thin sheet (5), the user can randomly attach the magic hook (9) to the surface of the non-textile fabrics layer (52) to adjust the tightness of the diaper (100).

Using the non-textile fabrics layer (52) to be the basic material of the rear thin sheet (5) can not only serve the economical purpose, but also solve the problem of material overuse to achieve the goal of saving resources. Moreover, utilizing the strength of the non-textile fabrics layer (52) itself to combine with the anti-leaking layer (51) and then utilizing the physical strength of the non-textile fabrics layer (52) to replace the usage of Loop to further lower the manufacturing costs of the diaper.

Furthermore, due to the combination of the anti-leaking layer (51) and the non-textile fabrics layer (52), the diaper manufacturer can avoid the problems such as misalignment, non-stickiness, misplacement and excessive glue because there is no Loop on the diaper, such that the quality of the diaper can be significantly increased.

Also, for the consumer purchasing the diaper (100), with the combination of the anti-leaking layer (51) and the non-textile fabrics layer (52), along with the magic hook (9), it is more convenient for the consumer because the area/surface which can be used for Loop is significantly increased so the consumer can randomly adjust the position and tightness whenever they want to provide a comfortable way for a baby's waist. Moreover, since the waist area of the diaper (100) can be adjusted in a broad range, the consumer can purchase any size of the diaper (100) so the flexibility is increased to avoid potential waste.

In another aspect, with the combination of the anti-leaking layer (51) and the non-textile fabrics layer (52), it reduces the usage of the materials to further protect the environment. A diaper may require several kinds of materials and several different methods to put the materials together. In modern high-speed manufacturing process, it is not an easy task to put together that many different materials to form a diaper. In other words, many process steps may be needed. Therefore, in addition to replacing Loop, the present invention can also simplify the manufacturing process to avoid mistakes occurred during the process, so that the yield can be increased and the usage of materials can be reduced to further achieve the goal of environmental protection.

Also, with the combination of the anti-leaking layer (51) and the non-textile fabrics layer (52), the strength of the conjugation is as strong as the replaced Loop. As to the diaper manufacturers, it is convenient to process and saves the materials. More importantly, the consumer does not feel uncomfortable and on the other hand, the present invention is closer to the consumer's style of using the diaper. Furthermore, with the combination of the anti-leaking layer (51) and the non-textile fabrics layer (52), it reduced the usage of the anti-leaking layer (51) with higher unit price so the costs are reduced as well.

The combination of the anti-leaking layer (51) and the non-textile fabrics layer (52) can be entirely or partially, wherein the partial combination is processed externally so that the designated portion of the diaper can be strengthened, and the strength is increased at the entire area of the diaper (100) or the small area on the Loop. The increase in strength is to strengthen and replace conventional Loop and the area and shape of the external process is not restricted by the drawing or area.

The surface of the rear thin sheet (5) can be one of the following: a velvet surface, a fabric surface or a rough surface. With the changes of the surface of the rear thin sheet (5), the present invention can be different in appearance and touch, as well as increasing the stickiness between the surface and the magic hook (9).

The surface of the non-textile fabrics layer (52) has a plurality of three-dimensional drawings (521) that are continuously and regularly formed on the surface of the non-textile fabrics layer (52). In other embodiments, the three-dimensional drawings (521) can be discontinuously and irregularly formed on the surface of the non-textile fabrics layer (52). In still other embodiments, the three-dimensional drawing (521) is one of the following: decorative patterns, dots, squares and regular stripes.

When the baby wears the diaper (100), the most outer surface of the diaper (100), namely the non-textile fabric layer (52) of the rear thin sheet (5), has the three-dimensional drawings (521) which are protruding or recessed, so the surface of the rear thin sheet (5) has protruding-recessed lines like other knit products, which can not only increase the diaper's sense of touch, but also provide better feeling to the parents especially when they hold the baby. The parents can directly feel gentle sense of touch from the three-dimensional drawings (521) on the non-textile fabric layer (52).

Furthermore, in addition to enhancing the sense of touch by adding the three-dimensional drawings (521), it can further strengthen the holding power between the anti-leaking layer (51) and the non-textile fabric layer (52), which can not only increase the aesthetic value, but also increase the conjugation between the non-textile fabric layer (52) and the magic hook (9) to increase the possibility of reuse.

On the surface of the anti-leaking layer (51)/non-textile fabric layer (52), there are either a combination of a regular mark (54) and a mark in the target area (55), or each mark individually. Even though it is already very convenient for the user by conjugating the magic hook (9) and the rear thin sheet (5), the user has been using conventional diapers for a long time. Therefore, in order to attract more consumers, in addition to adding more colorful marks, trademarks, or other marks with special design, the mark in the target area (55) can also be added to replace the area replaced by Loop. However, if the budget is not allowed, there can be no regular mark (54), nor the mark in the target area (55).

The anti-leaking layer (51) and the non-textile fabrics layer (52) are made by elastic materials and the elastic coefficients of the anti-leaking layer (51) and the non-textile fabrics layer (52) are different. Also, with the combination of the anti-leaking layer (51) and the non-textile fabrics layer (52), the present invention can reduce the baby's uncomfortable feeling and fit the baby's body so it is easier for the baby to move.

In one embodiment, the anti-leaking layer (51) is made of the following materials: breathable and non-breathable. More specifically, the anti-leaking layer (51) is made of one of the following materials: PP (polypropylene), PE (polyethylene), copolymerized composite materials. The material of the anti-leaking layer (51) can be determined by the application of the diaper (100).

Accordingly, with the combination of the anti-leaking layer (51) and the non-textile fabrics layer (52) along with the magic hook (9), the manufacturing costs, unsatisfactory yield ratio, and the material consumed are reduced. For the consumers, it is more convenient to use the diaper in the present invention. For the manufacturers, the present invention has more applicability, feasibility, functionality and industrial usage.

Having described the invention by the description and illustrations above, it should be understood that these are exemplary of the invention and are not to be considered as limiting. Accordingly, the invention is not to be considered as limited by the foregoing description, but includes any equivalents.

1. A diaper having an improved conjugation structure comprising:
a diaper including a front waistline segment located along vertical direction,
a rear waistline segment located along vertical direction facing the front waistline segment, and
a crotch segment located along horizontal direction and between the front waistline segment and the rear waistline segment,
wherein one side of the diaper contacting the skin has a liquid-penetrating surface sheet, and a rear thin sheet, which is not penetrated by liquid, is located at a side where the diaper does not contact the skin; and a liquid absorption unit extending between the front waistline segment and the rear waistline segment is located between the surface sheet and the rear thin sheet; and a pair of rear wings is extending from the liquid absorption unit toward the horizontal direction while a pair of side wing is extending from the liquid absorption unit toward the vertical direction,
wherein a magic hook is located at each side wing and extending towards the horizontal direction and the rear thin sheet is formed by an anti-leaking layer from the top and a non-textile fabrics layer which is conjugated with the anti-leaking layer entirely or partially,
wherein the strength to separate the magic hook and the rear thin sheet at 180 degrees is about 100 to 700 g/inch, and the shear stress at 180 degrees is above 1000 g/inch, and with the conjugation between the magic hook and the rear thin sheet, the user can randomly attach the magic hook to the surface of the non-textile fabrics layer to adjust the tightness of the diaper.

2. The diaper having an improved conjugation structure as defined in claim 1, wherein a surface of the rear thin sheet includes at least one of the following: a velvet surface, a fabric surface, and a rough surface.

3. The diaper having an improved conjugation structure as defined in claim 1, wherein a surface of the non-textile fabrics layer has a plurality of three-dimensional drawings.

4. The diaper having an improved conjugation structure as defined in claim 3, wherein the three-dimensional drawings are continuously and regularly located on the non-textile fabrics layer.

5. The diaper having an improved conjugation structure as defined in claim 3, wherein the three-dimensional drawings are discontinuously and irregularly formed on the surface of the non-textile fabrics layer.

6. The diaper having an improved conjugation structure as defined in claim 3, wherein the three-dimensional drawings include decorative patterns, dots, squares, and regular stripes.

7. The diaper having an improved conjugation structure as defined in claim 1, wherein the surface of the anti-leaking layer and/or the non-textile fabrics layer is one of the following or combination thereof: a regular mark and a mark in a target area.

8. The diaper having an improved conjugation structure as defined in claim 1, wherein the anti-leaking layer and the non-textile fabrics layer are made of elastic materials and the anti-leaking layer and the non-textile fabrics layer have different elastic coefficients.

9. The diaper having an improved conjugation structure as defined in claim 1, wherein the anti-leaking layer is made of a breathable material.

10. The diaper having an improved conjugation structure as defined in claim 1, wherein the anti-leaking layer includes at least one of the following materials: PP (polypropylene), PE (polyethylene), and copolymer composite materials.

11. The diaper having an improved conjugation structure as defined in claim 1, wherein the anti-leaking layer is made of a non-breathable material.
